(19) United States
(12) Patent Application Publication
(10) Pub. No.: US 2018/0036461 A1
(43) Pub. Date: Feb. 8, 2018

(54) DEVICE AND METHOD FOR HANDLING HUMAN BREAST MILK

(71) Applicant: MYMILK LABORATORIES LTD., Herzliya (IL)

(72) Inventors: Ravid SHECHTER USHPIZIN, Herzliya (IL); Sharon Rachel HARAMATI, Herzliya (IL)

(73) Assignee: MYMILK LABORATORIES LTD., Herzliya (IL)

(21) Appl. No.: 15/552,130

(22) PCT Filed: Feb. 26, 2015
(86) PCT No.: PCT/IL2015/050219

§ 371 Date: Aug. 18, 2017

(51) Int. Cl. A61M 1/06

(52) U.S. Cl. CPC A61M 1/064; A61M 1/0066

(57) ABSTRACT

A device for collecting human breast milk is provided. The device may include at least one evacuated container capable of connecting to at least one first container that is configured to receive expressed human breast milk. The at least one evacuated container mat be connected to the least one first container by a flow unit permitting communication of human breast milk from the first container to the evacuated container. Further provided are methods of collecting breast milk.

### TECHNOLOGICAL FIELD

The present invention relates to devices and methods for collecting, handling and using human breast milk.

### BACKGROUND

Breast milk is composed of nutrients, immune system components, probiotics and parabiotics, enzymes, hormones, growth factors as well as other cellular and molecular elements and is recommended for infant feeding. Breast milk can supply the baby with macronutrients and micronutrients, directly supporting the nutritional needs of the growing and developing baby.

In addition to its nutritional roles, human milk also provides the immune-immature newborn with immune defense mechanisms against viruses and pathogens and carries specific probiotica supporting nutrient absorption and protecting against harmful bacteria. Indeed, government health agencies in developing and developed nations promote exclusive breastfeeding and accordingly breastfeeding rates are rising globally.

Babies who cannot be fed directly from the breast are usually fed with expressed milk. At times, when mother's own milk is not available, milk from human milk bank is used. In some cases, human milk is supplemented with fortifiers to ensure nutrient adequacy. In a neonatal intensive-care unit (NICU), breast milk is the preferred nutrition for infants.

Devices and methods for collecting milk are described, for example, in U.S. Pat. No. 8,671,701 that describes method for storing human breast milk, preserving its natural flavor and nutritional content.

US patent application no. 2014/288466 describes a device for expression and collection of breast milk includes an actuatable assembly, a breast interface, and a tube.

US patent application no. 2012/265169 describes a device for collecting and administering fluid from a breast includes a container having a first end with a first opening for collecting fluid from the breast, and a second end with a second opening for administering the collected fluid.

### GENERAL DESCRIPTION

The present invention is based on the recognition that methodologies relating to diagnosis and utilization of human breast milk in, e.g., infant feeding, are at times cumbersome and prone to a variety of contaminations, which not only skew the diagnosis of breast milk but may also have detrimental effect on the fed human infant. The inventors of the invention disclosed herein provide an easy to use, bed-side device for breast milk collection, sampling, storing, and transport, of a sample which may subsequently be analyzed and diagnosed, e.g., for determining the quality of the expressed milk (analytics) or a medical condition, and determine components and concentrations thereof, and may also be used for feeding.

The devices and methods of the invention enable accurate collection of milk samples, optionally at different predetermined volumes and for different uses, while maintaining milk's cleanness, stability, natural flavor and nutritional content. As will be disclosed further below, in cases the milk sample is found to be deficient of any one essential component in the infant's diet, or for any other reason the sample may be enriched with any one of the essential components or with any other additive, therapeutic or non-therapeutic agents.

Human breast milk (used interchangeably with human milk or breast milk) is a unique body fluid, highly complex and greatly irreproducible liquid produced by the breasts (or mammary glands) of a human female, usually, for the purpose of infant feeding. The milk is considered as a non-homogenous emulsion, comprising a relatively large fat fraction, cellular components and non-cellular components, casein, whey proteins, carbohydrates and unique small molecules. When expressed, human milk is considered non-sterile. Any handling of an expressed milk sample becomes further challenging as it serves as a medium for microorganism growth. These features render the handling and storing, and any subsequent manipulation of breast milk samples unique as compared with other bodily fluids such as saliva, blood, urine, CSF and others.

The methodology disclosed herein allows expression of human milk and generation of pre-determined sample volumes of milk, permiting maintenance of the milk samples under sterile conditions preserving specific analyte content, while limiting external contamination. The expressed milk samples (portions) may be used for improved storage, feeding, analysis, determining the quality of the expressed milk, diagnosis or any combination thereof.

Beyond the benefits of each use, the invention provided herein has an advantage in its ability to maintain expressed human milk under conditions that enable accurate and reliable assessment of expressed milk quality, upon need, prior to or in parallel to infant feeding and as such optimize feeding.

In accordance with a first aspect, the invention provides a device and a method for collecting human breast milk employing such a device, the device comprising a first container (a first receptacle), and at least one further container (a further receptacle), at least one of said further containers being an evacuated container (container with a pressure less than atmospheric pressure). The “container” or receptacle may be any three-dimensional structure having at least a first end and a second end and having an inner surface defining a cavity extending between the openings at the first and the second ends along an axis.

The first container is selected to receive and hold (bear, carry) human breast milk expressed from a human breast. Exemplary and non-limiting designs of a device and a container for utilization in accordance with the invention are provided in the accompanying figures.

An exemplary container is depicted in FIGS. 1A to 1D showing schematic representations of such a first container and features thereof. FIG. 1A depicts a first container having a funnel-like shape or cone shape 12A that may be suitable for manual human milk expression. FIG. 1B shows a first container 12B comprising an element 16 that may be part of or connected to (adaptor) a breast pump and another element having a funnel-like shape or cone shape element 14. Elements 14 and 16 may form part of a breast pump. As exemplified, the first container of FIG. 1B may be suitable for human milk expression using a pump. FIG. 1C shows a first container having a cup shape 12C that may be suitable for manual human milk expression or for receiving milk from another container, for example a container (reservoir container) that was used for milk expression (manual expression or pump expression). FIG. 1D shows a first container having a cup shape 12D with a conical bottom 18. The first container in FIG. 1D may be suitable for manual human milk expression or for receiving milk from another container for example a container (reservoir container) that was used for milk expression (manual expression or pump expression).

In accordance with the present disclosure, the device may comprise at least one evacuated container, which directly or indirectly via another element or feature disclosed herein, is associated with the at least one first container. In some embodiments, the device comprises similar or different evacuated containers. When referring to different evacuated container it may be regarded as having at least one different feature (characteristics), such as but not limited to different shape, different structure, different size, different texture, different composition or manufacture, different color, and any other feature provided that the container is evacuated. The differences may be reflected in a part or a portion of the evacuated container. In addition and as described herein below, the differences between the evacuated container(s) may be reflected by the nature of an additive or in the amount (concentration) of the additive added to the evacuated container. In addition and as described herein below, the differences between the evacuated container(s) may be reflected by the structure of the evacuated container (the entire structure or part thereof).

Evacuation of the container can be for example during the manufacture step of the container. The at least one evacuated container is maintained in an evacuated form until such time when expressed milk flows into the container and increases the pressure in the container. As may be understood, the “evacuated” container is one from which ambient air, including oxygen was at least partially removed prior to use in accordance with the invention, and the pressure in the container is below atmospheric pressure. The evacuated container is maintained sterile.

The evacuated container may be mutatis mutandis an evacuated tube such as those utilized in blood collection.

Thus, in some embodiments, the device comprises at least one container suitable to receive and hold (bear, carry) human breast milk expressed from a human breast and at least one evacuated container. A method utilizing a device according to the invention allows collecting expressed milk in the at least one container and permitting, as explained herein below, spontaneous flow of the milk into the at least one evacuated container, which directly or indirectly is in liquid communication with the at least one container. In exemplary embodiments, the at least one evacuated container may be connected via a suitable adaptor unit to a breast pump or to a cup or a funnel used in manual expression of milk.

The device described herein also comprises means to allow flow communication of human breast milk from the first container to any one or more of the evacuated containers. The means to allow flow between the two receptacles is a “flow unit” which permits liquid communication from the at least one container to the e.g., evacuated container; namely flow/transfer of milk from an interior of the first container to an interior of the evacuated container via vacuum force.

In some embodiments, at least a part (portion) of the first container and at least a part (portion) of the evacuated container(s) are made of an inert material that does not interact with the human milk, does not affect the quality of the milk, does not contribute to its deterioration and which protects the milk from light or external contaminants In some embodiments, at least part of the first container and at least part of the evacuated container are made of a non-hazardous and non-toxic material. In some other embodiments, at least part of the first container and at least part of the evacuated container are made of a food grade material. The first container may be rigid, semi-rigid, flexible or any combination thereof. The evacuated container may be rigid, semi-rigid or of any material which may hold negative pressure and would not collapse under the evacuated conditions.

In some embodiments, the evacuated container is not of a flexible material.

In accordance with the present disclosure, at least the inner surface of the first container and the inner surface of the evacuated container are of a material that does not interact with human milk and is nontoxic. In some other embodiments, at least the inner surface is made of non-hazardous, food grade material.

In some embodiments, the first container and the evacuated container are molded plastic materials being of different shapes. In some embodiments, the first container and the second container may be of a material selected from or comprising glass, a variety of polymers, silica or any combinations thereof. The polymeric materials from which the containers may be made of may contain other substances to improve performance and/or reduce costs. Non-limiting examples of the polymeric are polyesters (PES), polyethylene terephthalate (PET), polyethylene (PE), high-density polyethylene (HDPE), polyvinyl chloride (PVC), polyvinylidene chloride (PVDC), low-density polyethylene (LDPE), polypropylene (PP), Polystyrene (PS), high impact polystyrene (HIPS), polyamides (PA) (Nylons), acrylonitrile butadiene styrene (ABS), polycarbonate (PC), polyurethanes (PU), blends thereof or any combinations thereof. In some other embodiments, the first container and the second container comprise PET, HDPE, LDPE, PVDC, PP, PS or HIPS.

In some other embodiments, the first container may be any receptacle capable of holding a liquid. In some embodiments, the first container may be a bottle, a jar, a bag, a jug, a straw, a cup, a tube or any other receptacle. In some further embodiments, the first container is a specimen cup.

In some further embodiments, the first container is a breast pump or part thereof.

In some embodiments, one or more of the first containers, the flow unit and the at least one evacuated container are for single use. In some embodiments, the first container and/or the flow unit are for multiple uses.

In accordance with the present disclosure, at least a part of the first container and at least a part of the evacuated container may be transparent or opaque or of any transparency, which transparency being selected based on the specific final use of the milk sample. In some embodiments, at least a part of the first container and at least a part of the evacuated container may be prepared from a material that enables preservation of light-sensitive analytes. In some embodiments, at least a part of the first container and at least a part of the evacuated container is prepared from a light protecting material. In some embodiments, the first container is at least partially transparent or has a transparent region which allows for observing the level (amount, volume) of the milk contained in the container.

As described herein, the first container is suitable for receiving and holding expressed milk. In some embodiments, the first container is adapted to be suitable for an expression process. The term “expressed milk” or “milk obtained by expression” as used herein denotes breast milk that has been taken from a woman's breast without having the baby suckle on the breast. The expressed milk may be obtained by any known method, such as but not limited to, hand expression (manual expression), or pump expression by a device such as a hand-operated pump or an electric pump.

In some embodiments, the openings at the first end and the second end of the first container permit fluid communication therethrough; namely are open. In some other embodiments, the openings at the first end and the second end of the first container are closed, but may be operable or transformable into an open state. In some embodiments, the opening at the first end of the first container is open and the opening at the second end of the first container is closed or visa-versa.

In some embodiments, the cavity defined by the first container is defined by at least a “tapering”, namely a gradual thinning or narrowing towards one end of a conical shape. In some other embodiments, the inner surface defining the first container cavity comprises a funnel-shape. In some further embodiments, the first container defines at least a funnel or cone-shaped tapered inner surface. In accordance with the present disclosure, the cone shape or funnel shape is not defined by straight lines (is curved). In accordance with the present disclosure, the cone shape or funnel shape may be defined by curved lines.

In some further embodiments, the cavity of the first container has a cylindrical shape. In some other embodiments, the first container has a “specimen cup” shape.

In some other embodiments, the cavity of the first container is characterized by at least a hemisphere shape (corresponding to substantially half of a sphere).

In some embodiments, at least a part of the first container has a funnel shape. In some further embodiments, at least a part of the first container has a cup-like shape. In some other embodiments, at least a part of the first container is a plastic bag. In some other embodiments, at least a part of the first container is a specimen cup. In some other embodiments, at least a part of the first container has a hemisphere shape.

In accordance with the present disclosure, the first end of the first container is denoted herein as “a first receiving end” and the second end of the first container is denoted herein as “a second delivering end”.

The first receiving end is the end of the first container being suitable for receiving (introducing) breast milk such that the first container, after receiving the expressed milk, through the first receiving end, holds (bears, contains) the milk. Similarly, the second delivering end is the end of the first container which is suitable for delivering (flow) breast milk into a further, e.g., evacuated container.

In some embodiments, the first receiving end and the second delivering end are both the same opening of the container. Thus, in some embodiments, sample may be introduced and withdrawn from the same opening of the container.

In some further embodiments, the first container is adapted for hand milk expression (manual). In some embodiments, the first receiving end of the first container has an opening suitable for hand milk expression (manually). In some further embodiments, the first receiving end of the first container has a rim shape. In some further embodiments, the first receiving end of the first container has a shape suitable for receiving milk sprinkles during manual milk expression from the nipple.

In some other embodiments, the first container is a part of or adapted to be a part of a breast pump such as a hand-operated pump or an electric pump. The first container may be anatomically contoured to fit on a woman's breast. In some embodiments, the first receiving end of the first container has a breast nipple enclosing (breast shield) to fit and operably suit a female breast. In accordance with some embodiments, the first receiving end of the first container is adapted to be suitable for pump milk expression. In some further embodiments, the first container comprises or is connected to an adaptor fitted to a milk pump. In some embodiments, the adaptor has a funnel-like shape.

The present disclosure is not limited to a specific type of breast milk pump and can be adjustable to any available pump. Non-limiting examples of breast pump include medela breast pumps, lansinoh, spectra and avent.

The device described herein comprises at least one evacuated container. When referring to more than one evacuated containers, it should be noted that the various evacuated containers may be similar or different as described herein. The differences in the evacuated containers provide the option of directing the at least one collected milk samples for various different applications, possibly different uses (feeding, analysis, diagnostics, analytical or any combination thereof) or for the same use but for different purposes (such as for diagnosis of different conditions or for analytical of different analytes or for feeding infants with different needs). At least one of the further containers is an evacuated container which is pre-evacuated.

The present invention is not limited to a specific feature of the evacuated container. As described herein, the evacuated container may have different characteristics such as but not limited to shape, structure, volume, composition of manufacture, color or any physical or chemical feature. In some embodiments, the evacuated container is in the shape of a cup or a tube. In some other embodiments, the evacuated container is an evacuated tube.

In some embodiments, the evacuated container has a volume of about 0.2 ml to about 60 ml, at times between about 1 ml to about 50 ml, at times between 3 ml to about 40 ml, at times even between about 6 ml to about 30 ml, at times between about 8 ml to about 20 ml, at times between 5 ml to about 10 ml, at times between 0.5 ml to about 10 ml.

In some other embodiments, the evacuated container has a draw volume of about 50 ml at times about 40 ml at times about 20 ml at times about 10 ml at times about 9.5 ml, at times about 4 ml, at times about 3 ml, at times about 2 ml at times about 1.5 ml at times about 0.5 ml.

The evacuated container in accordance with the present disclosure has a first end and a second end. In some embodiments, the openings at the first end of the evacuated container permit fluid communication. In some other embodiments, the openings at the first end and the second end of the evacuated container are closed, but may be independantly operable or transformable into an open state. In accordance with the present disclosure, the first end of the evacuated container is suitable (adopted) to receive milk from the second delivering end of the first container.

In some embodiments, the first end of the evacuated container may be a closed end. The first end of the evacuated container may be closed by a capping. The capping may be of or comprise of any material that on one hand allows insertion (puncturing) of elements, e.g., a needle, throughout and on the other hand is capable of sustaining the vacuum pressure, namely avoiding its collapse into the evacuated container. The material may be a flexible material such as a rubber material. The material may be able to sustain puncturing so it sustain a leak-tight and air-tight seal after disconnection from a “flow unit”. The capping at the first end may have different shapes such as T-like shape, namely having an upper surface located above the ending of the container and a lower surface located within the container. In addition, the closing may have a cylindrical shape, namely located within the container and having suitable means to ensure that the closing is capable of sustaining vacuum.

In accordance with some embodiments, the first end or the second end of the evacuated container are suitable (adopted) to deliver or allow flow of milk directly into a feeding system. The second end of the evacuated container may have different shapes such as a round shape or a funnel shape or a barrel-tip shape. The second end of the evacuated container may be closed but may be operable or transformable into open state upon need. As shown herein below, the second end of the evacuated container may be adapted to be in flow communication with gavage system (tubing) after is was filled with milk.

In the context of the present disclosure, the first container, the evacuated container or both may be empty containers or the first container, the evacuated container or both may contain at least one additive.

As used herein the term “additive” is used to denote any substance that may be added or contained in the first container, the evacuate container or both. The device according with the present disclosure may comprise several evacuated containers, each having the same additive or each having a different additive or each having a different concentration of the same additive. Also and in accordance with the present disclosure, the additive contained in the first container may be similar or different than the additive contained in the at least one of the evacuated containers. The additives may be used in any appropriate concentration ranging from about 0.00001% to about 90% of the final milk sample. The additives may be added to the evacuated container(s) prior to its use. Pre-filling of at least one evacuated container can be for example during the manufacture step of the container or prior to the evacuation step. In such a case and as described herein, an evacuated container may be selected for a specific purpose.

The at least one additive is not limited to a specific type and may depend on the specific purposed utilization. The additive may be a single or a combination which includes a plurality of different components. Non-limiting examples of additives include a preservative, a pharmaceutical such as a therapeutic agent or a drug or a component that is beneficial for an infant well-being (when the sample is utilized for feeding), a fortification agent such as a food fortifier, a filtration-aid agent, a homogenizing agent, or a quantification agent.

For the sake of brevity, the additives have been categorized into specific groups. However, as a person of skill would realize, one or more of the additives specifically or generically mentioned herein may be suitably categorized in different multiple groups.

The addition of the at least one additive may depend on the intended use of the milk collected in the at least one evacuated container. If the additive is used to maintain or prolong stability and shelf-life of the milk sample, the at least one additive may be a preservative. If the collected milk is used for further diagnosis and/or for determining the quality of expressed milk (analytical), the additive may be selected for example for the purpose of homogenization and preservation of the milk sample and/or for quantification of at least one tested analyte (analyzed, determined) in the milk sample.

In some embodiments, the at least one additive is selected from a filtration-aid agent, a homogenizing agent, a quantification aid agent and a preservative.

In accordance with some embodiments, the at least one additive may be a food grade additive. In case the collected milk is used for infant feeding (directly or for use in a milk bank), the additive may be selected for the purpose of homogenization, preservation, food fortification (e.g., protein, minerals, vitamins, sugars, commercially available fortifiers, thickening substances and others), or for therapeutic purposes, e.g., treatment or prevention of a disease or disorder (e.g., drug, antibiotics, protein, minerals, sugars, insulin and others). In some other embodiments, the at least one additive is selected from a filtration-aid agent, a homogenizing agent, a quantification aid agent, a preservative, a pharmaceutical substance (drug) and a fortification agent (such as milk (food) fortifier). In some further embodiments, the at least one additive is a therapeutic agent or food fortifier.

In accordance with the present disclosure, additives present in the first container may flow with the expressed milk into the evacuated container. Alternatively, the evacuated container may contain an amount of the at least one additive, as defined.

As noted herein, the at least one additive, may be from the same group of additives or from different groups/subgroups of additives, namely to a combination of different additives (possibly from different groups/subgroups). The group of additives may encompass various subgroups.

The “homogenizing agent” may influence the homogeneity of a milk sample. Non-limiting examples include surfactants (ionic or non ionic) or emulsifiers.

The “filtration aid agent” may assists (aids) in filtration. The agent may assist in maintaining or allowing flow of milk from the first container into the evacuated container. The filtration aid agent may assist in allowing flow of possibly a larger amount of milk than without such an agent, from the first container into the evacuated container and reduce possible clogging of the filtration media. In some embodiments, the first container comprises a filtration aid. The filtration aid may be an inert material. Non-limiting examples include diatomaceous earth (DE), perlite, cellulose, asbestos, agricultural fibers, saw dust and paper fibers.

It should be noted that filtration and/or homogenization according with the invention may be done according with any known method in the art and is not limited to the use of filtration aid agent and/or homogenizing agent.

The “preservative” may be used to prevent decomposition of the milk sample that may be caused by microorganisms, e.g., microbial growth, or by a chemical condition or any other condition affecting the conditions under which the milk sample is maintained, e.g., temperature, humidity, exposure to light, etc. In some embodiments, the preservatives are an antibacterial agent, a bacteriostatic agent (or bacteriostat), a bactericide, an enzymatic activity inhibitor, a stabilizer, a photostabilizer, pH adjuster, buffer, an antioxidant or a material capable of stabilizing fungal count in human milk Non-limiting examples include antibiotics and ferrous sulfate. In some embodiments, the preservatives are food grade preservatives.

The antibiotics or antibacterial agents are agents used to kill or inhibit the growth of bacteria. Non-limiting examples of an antibacterial agent include for example bronopol, EDTA, sodium borate, and sodium formate, or pH stabilizer such as ammonium acetate, alcohols such as ethanol, azide and alike. The term antibacterial agent may include also antifungal agents. The use of the term may not be compatible with bacterial culture. In some embodiments, the antibacterial agent is bronopol. In some other embodiments, the antibacterial agent is sodium borate. In some further embodiments, the antibacterial agent is sodium formate.

The bacteriostatic agent is a biological or chemical agent that stops bacteria from reproducing, while not necessarily harming them otherwise. Bacteriostats are often used in to prevent growth of bacteria on surfaces for example plastic surfaces. Non-limiting examples of bacteriostats include thiomersal, boric acid, bronopol, sodium borate, and sodium formate, or pH stabilizer such as ammonium acetate, alcohols such as ethanol, azide and alike. The bacteriostatic agent may encompass bacteriostatic antibiotics that limit the growth of bacteria. Non-limiting examples of bacteriostatic antibiotics includes tetracyclines, sulfonamides, spectinomycin, trimethoprim, chloramphenicol, macrolides, lincosamides, clindamycin, ethambutol, nitrofurantoin, novobiocin, tigecycline or oxazolidinone. In some embodiments, the bacteriostat is boric acid. In some embodiments, the bacteriostat is sodium borate or sodium formate.

The bactericide is a substance that kills bacteria, including for example disinfectants, antiseptics (including but not limited to alcohol, azide, formaline and alike), or antibiotics.

The antioxidants inhibit oxidation and may be important for oxygen sensitive analytes (for example vitamins and fatty acids). In some embodiments, antioxidants may include thiols, ascorbic acid, or polyphenols. Non-limiting examples include but not limited to butylated hydroxytoluene (BHT), butylated hydroxyanisole (BHA), pyrogallol, hydroquinone, EDTA or ascorbic acid (vitamin C). As detailed above, the antioxidants may be used in a concentration that is effective for inhibiting (reducing) oxidation. In some embodiments, the at least one antioxidant is added at a concentration of about 0.001% to about 5%. In some other embodiments, the concentration of the at least one antioxidant is between about 0.01% to about 1%. In some embodiments, the antioxidant is BHT. In some other embodiments, the antioxidant is ascorbic acid.

The “quantification aid agent” may be an internal standard for specific quantifications based on the analyte to be measured and the method of intended detection. In some embodiments, the quantification aid is an internal standard for water soluble components. In some further embodiments, the quantification aid is caffeine or caffeine analogs.

The “food fortification” or “food fortifier” and the “pharmaceutical agent” are added to human breast milk for enriching (strengthening) the milk content. The “food fortification” or “food fortifier” is not limited to a food product or a food component and may encompass any agent that is safe for use in food (non-toxic) such as a food grade additive for example an additive used in the food industry.

The food fortification agent or food fortifier and the pharmaceutical agent may increase or replenish the content of milk irrespective of whether they were originally present in the breast milk and as such may include any agent that was not originally present in the breast milk.

Non-limiting examples of food fortifier include proteins, vitamins, fatty acids, amino acids, hormones, minerals, probiotic bacteria, carbohydrates, sugars, enzymes, lipids, immunoglobulins, lipoproteins, commercially available breast milk fortifiers, thickening substances. The food fortifier may be used to enrich the nutritional content of the expressed milk.

Non-limiting examples of pharmaceutical agents include drugs (medicaments) or any agent that is considered beneficial for the infant well being. Pharmaceutical agents may be any small molecule, an antibody or parts thereof, protein or peptide, each or in combination may be utilized to treat or prevent a disease or disorder or otherwise improve the well being of the infant.

In accordance with some embodiments, the additive may be a combination of preservatives. In some embodiments, the combination comprises at least one antioxidant and at least one bacteriostat. In accordance with some embodiments, the combination comprises at least one anti oxidant and at least one antibacterial. In some further embodiments, the combination comprises at least one anti oxidant and at least one antibacterial and at least one bacteriostat.

As described herein, the device comprises at least one evacuated container, each may comprise a similar additive, a different additive or a different concentration of a similar additive or alternatively the at least one evacuated container may be free of additives (namely has no additive).

The evacuated containers comprising at least one additive suitable for the purposes disclosed herein are contemplated as aspects of the present invention. Thus the invention provides evacuated tubes comprising one or more additives, as used herein, the at least one additive being suitable for use in combination with human breast milk.

The invention further provides a solution, emulsion or combination comprising an amount of human breast milk and at least one additive as disclosed herein. In some embodiments, the additive is selected amongst additives useful for maintaining stability and shelf-life of the milk, such an additive may be e.g., a preservative, a homogenizing agent, a quantification aid agent. In other embodiments, the additive is selected to enrich the milk with at least one therapeutic or food additive, such as e.g., a drug, a fortification agent, a food fortification agent, and others.

As noted above, the device according with the present disclosure comprises a flow unit. The flow unit in accordance with the present disclosure is suitable to allow communication and essentially leak-free flow of milk from the first container to the evacuated container. In some embodiments, the flow unit allows flow of milk from the interior surface of the first container to the interior surface of the second container by vacuum force. In some further embodiments, the flow unit may be composed of several components essentially permitting adaptation (communication) between the first container and the second container.

In accordance with some embodiments, the flow unit comprises means to allow flow of milk into the second container. In some embodiments, the flow unit is adapted to be inserted (introduced) into the first end of the evacuated container. In some further embodiments, the flow unit is suitable to puncture the evacuated container in order to allow flow of milk. In accordance with some embodiments, the second end of the flow unit comprises means to puncture the first end of the evacuated container and possibly a “tube holder”. In some embodiments, the flow unit comprises a needle.

The term “adapter” is used herein to denote means that enable physical attachment of two different objects. In accordance with some embodiments, the adapter may be a male-female taper. In some embodiments, the first end of the flow unit comprising a male-taper fitting and connects to the second delivering end of the first container comprising a mating female part or vise-versa.

The term “tube holder” is used herein to denote means that enable physical holding of one object by the other. In some embodiments, the second end of the flow unit comprising a “tube holder” that accepts the first end of the evacuated container during, for example, puncturing.

The flow unit may be an independent unit that is suitable for connecting (joining) to at least the second delivering end of the first container and to at least the first end of evacuated container and as such allow flow of milk from the first container to the second container. In case a flow unit is not present (namely is absent), no flow of milk exists between the first container and the second container. In some embodiments, the flow unit may be connected to the second end of the first container upon need or use. In some further embodiments, the flow unit can be connected to the first end of the evacuated container upon need or use.

The flow unit may be an integral part of the first container and/or part of the second container or both.

Reference is now made to FIG. 2 showing a representative example of a flow unit 30 including an adaptor 32 with rubber boot 34 covered needle 36. The flow unit may comprise also a vacuum tube holder 38, with luer lock connector 40. The flow unit may be integrated with a filter 42.

In some embodiments, the adaptor 32 enables leak-free connections between a male-taper fitting and a mating female part as described herein.

In accordance with the present disclosure, the flow unit permits transfer of different volumes of milk from the first container to the evacuated container. It should be noted that the flow unit is by no way limiting of the amount of milk transferred from the first container to the second container. In some embodiments, the flow unit may transfer from about 0.5 ml of milk up to 50 ml, at times from about 1 ml to about 20 ml, at times between 5 ml to about 10 ml.

In some embodiments, the flow unit may be suitable to transfer from about 1% of milk volume up to 100% of the milk volume from the first container. In some other embodiments, the volume (amount) of milk transferred from the first container to the second container is pre-determined.

Without wishing to be bound by theory, it is suggested that using a filter enables selective transfer of milk components, namely reduced fat fraction (skim milk) and cell free (bacteria and somatic cells) milk in order to obtain fractionated milk. Thus, in some embodiments, the device described herein may comprise at least one type of a filter.

As disclosed herein and in accordance with the present disclosure, for detection of analytes found in whey fractions, for example water soluble vitamins and minerals, electrolytes, enzymes, nucleic acids, proteins, immunoglobulins, human breast milk from the first container may be subjected to filtration before flowing into the evacuated container so that a sterile (bacteria and cell free) fat free fraction is introduced into the evacuated container for further use. As also described herein, the collected milk samples may be analyzed on site or transported for further analysis.

As also described herein, filtration may be achieved by using a filter placed between the first container and the evacuated container or the filter is part of the flow unit. The inventors have shown that when attaching a 0.45 syringe filter between the first container and the flow unit and connecting the evacuated container to the flow unit, the raw milk sample in the first container was drawn by vacuum force (generated solely by the vacuum in the evacuated tube) through the filter to the evacuated container, obtaining a clear milk filtrate in the evacuated container.

Further and as described herein, homogenizers, such as for example emulsifiers may be introduced into the first container, the evacuated container or both to obtain homogenous sample that may be useful for milk sample transport, representative sampling (to the evacuated container or in the laboratory) and may assist in reliable analysis.

In accordance with the present disclosure, the filter may be any filter known in the art. In some embodiments, the filter is any one suitable for filtration of a biological sample. In some other embodiments, the filter comprises filtration aid as pre-filtration media or membrane filter. In some embodiments, the filter has pore size of between about 0.2 to about 0.45 micrometer. Non-limiting examples include filter paper/membrane, such as cotton wool, filter paper, glass wool, cellulose acetate, nylon, nitrocellulose, Polyvinylidene fluoride, polyvinylidene difluoride (PVDF), Polyvinylpyrrolidone (PVP) and polyether sulphonate.

In some embodiments, the flow unit may be at least partially integrated with a filter. In some further embodiments, the filter is present at the first end of the flow unit. In some the filter is located between the delivering end of the first container and first end of the flow unit.

Reference is now made to FIGS. 3 to 5 showing schematic representations of the device in its ready for use configuration. For simplicity, similar elements in FIGS. 3 to 5 are provided with similar numberings (at times in the hundreds) as in FIGS. 1 and 2.

FIG. 3 depicts a device 10 according to some embodiments of the present disclosure comprising a first container 112, an evacuated container 50 and a flow unit 130. The first container 112 has a first receiving end 52, a second delivering end 54, and an inner surface 56 defining a funnel-like or cone shape. The evacuated container 50 has a first end 58 and a second end 60, and an inner surface 62. FIG. 3 also shows representative embodiments according to which the first container 112 may be placed within an adjustable solid structure 64 providing stability for the first container connected only to the flow unit without the second container. The device shown In FIG. 3 may be suitable for manual milk expression.

For simplicity, similar elements in FIG. 4 are provided at times with similar numberings (in the hundreds) as in FIGS. 1 to 3.

FIG. 4 shows a device 10, first container 112, an evacuated container 50 and a flow unit 130. The first container 112 has a first receiving end 152 and a second delivering end 154, and an inner surface 156 defining a funnel-like or cone shape. The first container 112 comprises an element 116 that may be part of or connected to (adaptor) a breast pump and may be suitable for human milk expression using a pump and a funnel-like shape or cone shape element 114. The evacuated container 50 has first end 158 and a second end 160, and an inner surface 162.

FIG. 5 shows a schematic representation of a device 10 comprising a first container having a cup shape 112, an evacuated container 50 and a flow unit 130. For simplicity, FIG. 5 shows a cup having a shape as shown in FIG. 1D; however, the same holds for a cup 16 as shown in FIG. 1C. The first container 112 has a first receiving end 152 and a second delivering end 154, and an inner surface 156 defining a cup shape. In the schematic representation depicted in FIG. 5, the first receiving end 152 and a second delivering end 154 partially overlap. The evacuated container 50 has first end 158 and a second end 160, and an inner surface 162.

FIG. 6 shows a specific representation of a device depicted in FIG. 3 comprising a first container 112 having a hemisphere shape and an embedded transfer unit.

As described herein, the device is for use in human breast milk collection. As also described herein, the device allows flow of milk from the first container to the evacuated container. The evacuated container filled at least partially with human breast milk, is denoted herein as collected milk sample.

The at least one collected milk sample may have a closed first end and/or a closed second end. Both the first end and the second end may be operable or transformable into open states.

After certain volume of milk has flown from the first container to the evacuated container, the evacuated container may be separated from the flow unit to obtain a collected milk sample. In some embodiments, the closing at the first end of the evacuated container is made from a material (such as flexible material) such that after the flow unit or part thereof is removed, the evacuated container remains sealed (air tight and/or liquid tight).

The collected milk sample may be further used, for example for infant feeding, determining the quality of the expressed milk (analytical) and/or diagnosis. In accordance with the present disclosure, at least one of the collected milk samples may be used for diagnosis, at least one of the collected milk samples may be used for infant feeding or at least one of collected milk samples may be used for determining the quality of human breast milk.

In some embodiments, the device is used for feeding. In some embodiments the collected milk samples may be used for direct gavage feeding. In some embodiments, the collected milk samples may be further divided into sample aliquots.

As described throughout, the device described herein may be used in a method for breast milk collection, storing and transporting the expressed milk. In accordance with some other aspects, the present disclosure describes a method for using a device as described herein for collection of human breast milk comprising (a) providing a first container that is at least partially filled with human breast milk and (b) allowing the human breast milk to flow from the first container to a second evacuated container, thereby obtaining the evacuated container at least partially filled with human breast milk (denoted herein as “collected milk sample”). It should be noted that in accordance with the present disclosure, when referring to an evacuated container being at least partially filled with human breast milk, it is to be understood that the tube comprising the milk is no longer in a vacuum state as before. Namely, flow of milk from the first container to the second evacuated container resulted in reduced vacuum in the second container. Therefore and in accordance with the present disclosure, the term collected milk sample as used herein denote samples of milk collected within tubes that were evacuate prior to the flow of milk from the first container into them or to samples of milk collected within tubes that were maintained in a higher vacuum prior to the flow of milk from the first container into them.

In some other embodiments and as shown in representative FIGS. 3 and 4, at least part of the first container is further from gravity as compared to the evacuated container. In some other embodiments, at least a part of the first container is closer to gravity. In some other embodiments, at least a part of the first container is located adjacent the at least part of the evacuated container.

A first step in the method disclosed herein comprises providing expressed human milk in a first container via a first receiving end. In some embodiment, the human milk is provided (obtained) in the first container during an expression process. In some other embodiments, the human milk is obtained in the first container after expression. In some other embodiments, the human milk is obtained in the first container from a reservoir of expressed human milk. In accordance with some embodiments, at least part of the inner surface (cavity) of the first container is filled with human breast milk. It should be noted that the milk provided in the first container as described herein in the first step may refer to an ongoing process of milk expression (manual or pump).

A second step of the method disclosed herein comprises allowing flow of milk from the delivering end of a first container to at least one evacuated container via a flow unit. For simplicity, at times when the evacuated container comprises breast milk (namely after flow of milk from the first container into the evacuated container), it is also denoted herein as “collected milk sample” and as described herein, is characterized by a different vacuum, e.g. lower vacuum compared with the vacuum of the evacuated container prior to being filled with milk (flow of milk from the first container). In some embodiments, the method may be used for collection of at least one collected milk sample at different or similar volumes (amount) of milk. In some embodiments, the at least one collected milk sample may be collected sequentially at one time point (one after the other) or at different time points (possibly after testing or at different feeding times). It should be noted that a device depicted in FIGS. 3 to 5 may be used at times for replacing several evacuated containers one after the other for obtaining several collected milk samples. The collected milk sample may be collected for different uses. In addition, the several evacuated containers may be different thereby obtaining different collected milk samples. Also, in accordance with the present disclosure, a first container may be replaced upon need.

The collected milk sample may be used immediately, or stored for further use, as ambient temperature sample, refrigerated sample or frozen sample.

Thus, in accordance with some further aspects, the present disclosure also encompasses collected milk samples.

In accordance with some embodiments and as described herein, the evacuated container may comprise additives.

In accordance with some embodiments, the method described herein may further comprise infant feeding and/or diagnosis and/or determining expressed milk quality (analytical). In accordance with some other embodiments, the method described herein may further comprise infant feeding. In accordance with some other embodiments, the method described herein may further comprise diagnosis and/or determining expressed milk quality (analytical).

The technology being the subject of the present invention, provides tools assisting or improving further “determining the quality of expressed milk” namely to provide quantitative and qualitative analysis of the expressed milk, by determining (detecting, measuring, testing) the presence and/or amount of at least one analyte in human breast milk. As used herein the term “analyte” refers to an endogenous analyte, namely a naturally occurring component in breast milk or an exogenous component. In the context of the present disclosure, the term “diagnosis” denotes determining markers for disease or condition in the mother, namely maternal assessment.

It should be noted that the fact that the method described herein provides means for an optional method step in which the collected milk samples are further subjected to diagnosis and/or determining the quality of expressed human milk is extremely important. Owing to the unique features of the device and method developed herein, diagnosis and/or determining the quality of expressed human milk may be done prior to or in parallel to infant feeding in order to provide a full assessment of the maternal condition, milk condition, or both for the purpose of infant feeding. For example, determining the quality of the expressed milk may indicate the need for increasing the amount/concentration of at least one analyte to a milk portion for baby feeding, for instance by utilizing a fortifier-containing first container or a fortifier-containing evacuated tube used for feeding, or increasing the maternal intake of the nutrient to increase its level in her milk.

As noted above, the collected milk samples may be used per se for feeding. In some embodiments, the collected milk samples are themselves milk reservoirs for subsequent infant feeding. Feeding may be by gavage feeding of infants (newborns).

In some embodiments, the evacuated container may be directly connected to a gavage feeding system (tubing). The evacuated container may be connected to gavage tubing via the first end or the second end.

In some embodiments, the first end of the evacuated container is connectable to a gavage system via an adaptor which comprises at least one needle designed to puncture the closing at the first end and allow direct flow of milk to the infant being fed (gavage). In order to allow flow of milk, it is required to allow entrance of air into the container. Air may flow into the container by introducing an additional needle through the adaptor. In some embodiments, the two needles of the adaptor may be inserted through the first end of the evacuated container. The first needle may be inserted into the evacuated container and may be directly connectable to the feeding system (tubing) such that it enables expressed milk to progress (flow) by G force. The second needle may be used as a venting needle or as a source for injection of air and/or gas into the evacuated container.

In some further embodiments, the adaptor comprises one needle that is used for direct flow of milk to the gavage system. Flow of air may be achieved by partially or fully opening a least a part of the second end, which is produced (molded) with a vent for introducing air to allow flow (vent kept capped until feeding).

In some embodiments, the second end of the evacuated container is connected to a gavage system via suitable means allowing direct flow communication between the second end and the gavage tubing. The second end of the evacuated container may have any shape suitable in order to allow flow communication, such as funnel shape or barrel-tip shape. In order to allow flow of milk, it is required to allow flow of air into the container (spontaneous or active), or alternatively, actively forcing the milk sample into the gavage system by sliding the rubber stopper within the barrel toward the feeding end. Flow of air may depend on the structure of the closing and may include partially or fully opening at least a part of the closing at the first end or the second end. Alternatively, the rubber stopper may be pressed by a connectable handle following releasing, for example, a safety hatch.

Reference is now made to FIG. 7A to 7C showing schematic representations of evacuated containers that may be used for feeding.

FIG. 7A depicts an evacuated container 200 connected to an adaptor 220. The adaptor includes a first needle 240 designed to allow direct flow of milk to the infant being fed (gavage) and a second needle 260 allowing entrance of air. The first end of the evacuated container is directly connected to a feeding gavage tubing 280 for infant feeding. In accordance with some embodiments, the adaptor 220 has unique features as it enables flow of milk from the collected milk sample to the gavage feeding tubing and flow of air into the collected milk samples. Flow of air is required in order to allow flow of milk for further use such as for direct infant feeding.

FIG. 7B depicts an evacuated container 300 with a second end having a funnel shape or a barrel-tip shape 320 which is maintained capped at all times until feeding. In accordance with some embodiments, the flow of milk from the second end to a feeding tubing 340 is possible after opening a capping at the first end of the evacuated container 360.

FIG. 7C depicts an evacuated container 400 with a second end having a funnel shape or a barrel-tip shape 420 which is maintained capped at all times until feeding. In accordance with some embodiments, the flow of milk from the second end of the evacuated container to a feeding tube 440 is possible after opening a safety catch 480 and connecting a handle 460 for manual or mechanical press. Upon removal of the safety catch, the cap (rubber cap 500) may slide within the container towards the second end of the container. When using an evacuated container as depicted in FIG. 7C, it should be noted that the first end is maintained closed (capped), for example with a rubber cap that is resistant to collapse and may be held with a safety catch 480.

The evacuated container may be designed to be directly connected to a gavage feeding system. This may be done, for example, by an appropriate adapter connected to the feeding tube.

As noted herein above, the evacuated container may be adjusted and/or manufactured to comply with requirements of infant feeding with respect to the structure and to the additives. In some embodiments, the evacuated container comprises different means for excretion (flow) of expressed milk for infant and for flow of air to the collected milk sample. For example, the evacuated container may be structured to connect to an adaptor (equipped) with at least one needle, at times two needles, or directly to common gavage tubing system.

In the gavage system adaptor, the milk flow needle may be a short needle as to allow maximal feed flow and minimize tube leftovers. A needle used for air flow may at times be long to allow air flow and minimize milk leakage and air flow through the milk that may cause oxidation of milk components.

The infant to be fed from a milk reservoir according to the invention may be any infant feeding on its mother's milk or may be an infant suffering, for example, from weak suckling, uncoordinated suckling and swallowing, respiratory distress, tachypnea, or repeated apneic spells. Breast milk given to babies and especially to early born may not be fully compatible to their nutritional need as it may lack certain nutrients such as proteins, vitamins and minerals. As such, monitoring the level (amount, concentration) of analyte in milk is important in order to be able to either provide food additives which are required for growth or avoid using the expressed milk in case it comprises analytes that may cause damage to the infant. In some embodiments, in order to enrich the expressed milk content, one can use first container containing additives, at least one evacuated container containing additives or both. In addition and as described herein appropriate additives may be a food grade additive such as but not limited to macronutrients, vitamins and minerals, fatty acids, proteins, enzymes, immunoglobulins and the like.

The additives may be prefilled, and the adequate container may be selected subsequent to a determination that the milk is absent of a particular, e.g., nutrient or analyte, or that the concentration of the e.g., nutrient or analyte is present at an insufficient or otherwise low concentration. Or upon need (medical or other).

The human milk collected as described herein may be further used in a method for non-invasively sampling in order to provide indicative properties for the condition of both mothers and their babies. Each mother's milk is unique in its composition, owing to cellular components as well as variability of non-cellular components and certain nutrients and as such has individual health consequences for the breastfed infant, mostly health promoting, but some health compromising to the infant (pathogens, pollutants, drugs etc.).

In accordance with some aspects, the method described herein may further provide analysis to the expressed human breast milk. Analysis may include determining the quality of the collected expressed human breast milk. Further, analysis may include determining maternal diagnostic markers in the expressed milk.

The human breast milk sample, e.g., obtained in accordance with the invention may be subjected to any assay known in the art (including but not limited to liquid chromatography (LC), gas chromatography (GC), Immunoassays (e.g ELISA), chemiluminessence (e.g., ECLIA) colorimetric reactions, spectroscopy, ion selective electrodes, microbiological assays, bacterial cultures) for determining the quality of the expressed human breast milk, such as obtaining a measure for the presence or absence of at least one tested analyte or a determining a change in the level of the at least one analyte in the expressed milk. The change may be any one of deficiency, presence or excess in at least one analyte.

The present invention may encompass pre-treatment steps of the expressed milk samples such as separation methods or saponification or extraction that may be required prior to determining the quality of the expressed human breast milk.

Determining the quality of the expressed human breast milk may be determining freshness of the tested milk.

The quality of expressed milk may be evaluated by studying a variety of components such as breast milk endogenous components. For example, micronutrient, macronutrients (for example carbohydrates, lipids, proteins), calories, bacteriological composition, fat soluble vitamins (such as fat soluble vitamins A, D, E and K), water soluble vitamins (such as water soluble vitamins B (for example B1, B2, B3, B6, B12 and C), minerals (for example Iodine (I) Selenium (Se) Zinc (Zn), fatty acids, proteins, amino acids, nucleic acids (DNA, RNA), enzymes, immunoglobulins, hormones, electrolytes (for example sodium (Na), potassium (K) or chloride (Cl), calcium (Ca), Magnesium (Mg) or Phosphorous (P)) or any of the like.

The fatty acid may be any fatty acid, and specifically fatty acids that are usually found in human breast milk including but not limited to long chain poly-unsaturated fatty acids (LC-PUFA) or trans-fatty acids. The fatty acids in accordance with the present disclosure may include omega-3 fatty acid or omega-6 fatty acid. Non-limiting examples of fatty acids include but not limited to Arachidonic acid (AA or ARA), Docosahexaenic acid (DHA), Linolenic acid (LA), Eicosapentaenoic acid (EPA), α-Linolenic acid (ALA) or gamma-linolenic acid (GLA).

In addition, determining the quality of the expressed milk may suggest that the expressed milk should not be used, for example, in case the expressed milk comprises a specific hazardous analyte or an analyte at a concentration that may be dangerous to the infant. Non-limiting examples include drugs of abuse, medicinal drugs, recreational drugs, trans-fatty acids, hazardous levels of vitamins and minerals, pesticides, pollutants and pathogens.

The method described herein provides collected expressed milk samples that may be further used for diagnosis, prognosis and monitoring of a condition in the mother. The diagnosis may utilize any known diagnostic method in the field or any combination thereof. For example, the human breast milk sample, e.g., obtained in accordance with the invention may be subjected to any assay known in the art, for obtaining at least one parameters associated with the condition; and determining the presence or absence of said condition, e.g., with respect to a predetermined standard value. The diagnostic using the collected milk samples may be used to identify and monitor various health conditions in the mother, including but not limited to infectious or non-infectious inflammation such as mastitis or appropriate or delayed lactogenesis 2, subclinical mastitis and/or weaning, and malignancies including but not limited to breast cancer.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A to 1D show a schematic representation of a first container in accordance with some embodiments of the invention.

FIG. 2 shows a schematic representation of a flow unit in accordance with some embodiments of the invention.

FIG. 3 shows a schematic representation of a device according with some embodiments suitable for manual expression milk.

FIG. 4 shows a schematic representation of a device according with some embodiments suitable for milk pump expression.

FIG. 5 shows a schematic representation of a device according with some embodiments suitable for milk reservoirs.

FIG. 6 shows a schematic representation of a device according with some embodiments.

FIGS. 7A to 7C show a schematic representation of an evacuated container in accordance with some embodiments of the invention.

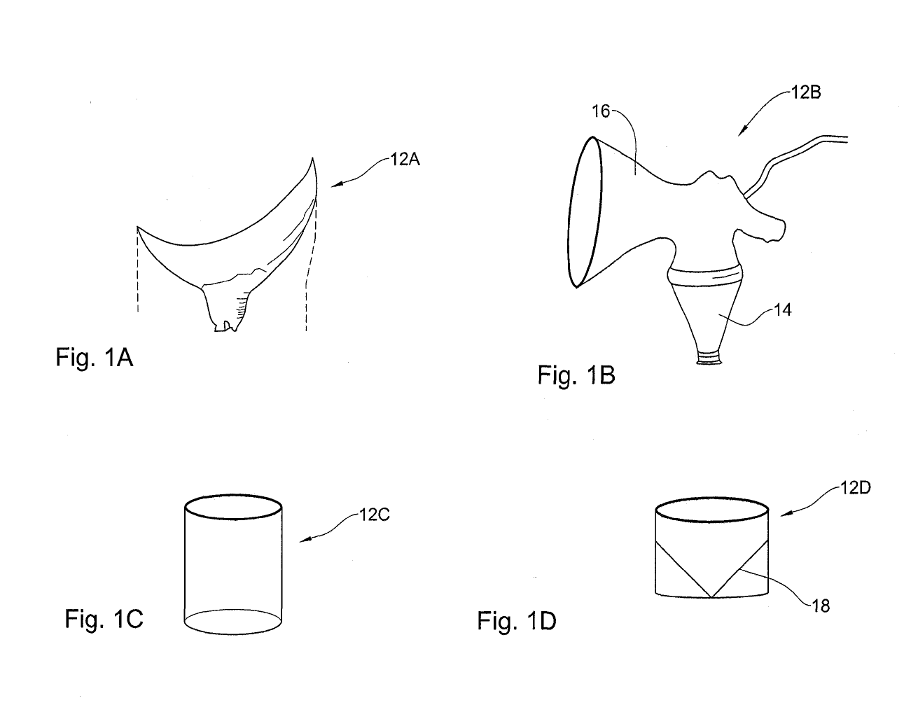

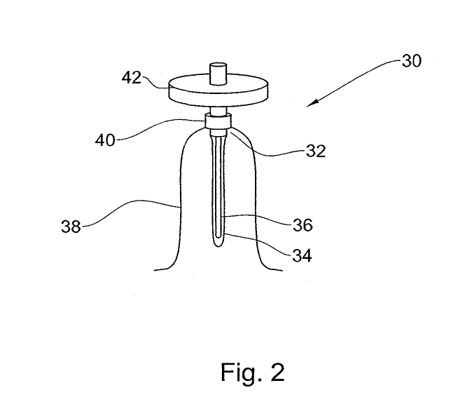

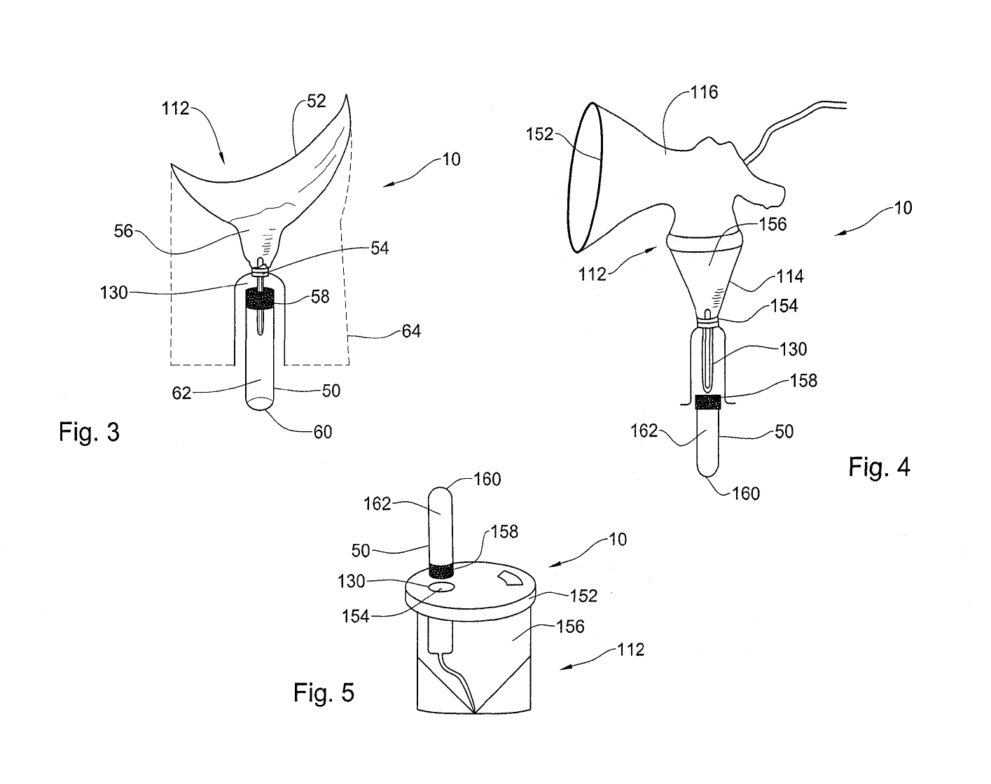

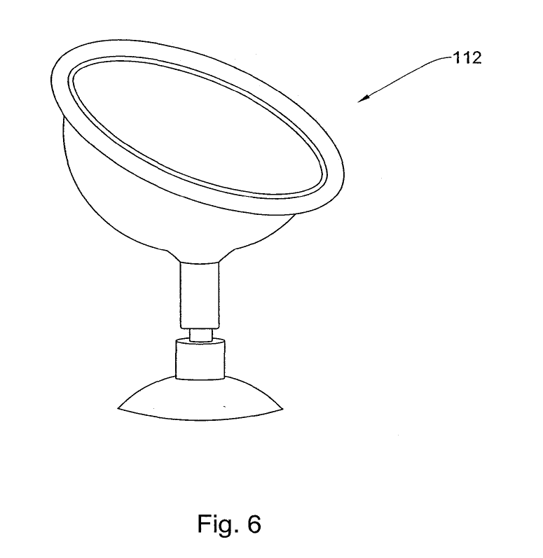

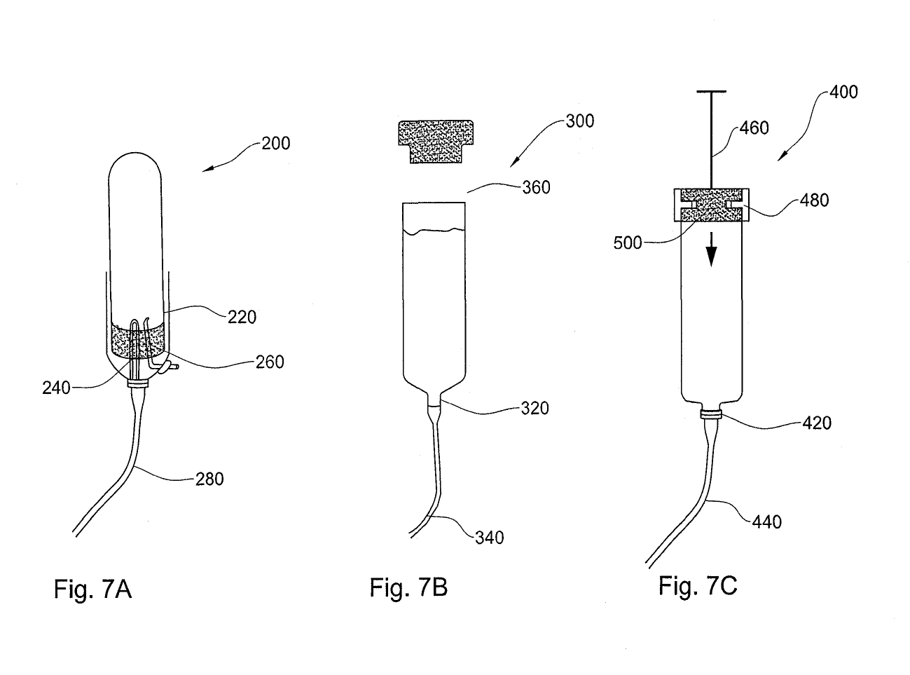

### DETAILED DESCRIPTION OF EMBODIMENTS

### NON-LIMITING EXAMPLES

### Example 1

A mother to a NICU hospitalized premature infant is expressing 30 ml of milk manually at home or in the medical facility into a first container to be transferred to a vacuum tube. Mother needs to divide the milk into 4 different tubes, three portions of 8 ml portions for direct gavage feeding of her pre-term baby, and one portion of 2 ml tube for macronutrient testing to ensure adequate caloric intake.

Evacuated tube that will be used for Feeding is pre-filled with powdered human milk fortifiers or medicine and the evacuated test tube is pre filled with sample preservatives and stabilizers. The evacuated sample tubes are inserted by the mother into the transfer device and the milk is forced into the evacuated tubes (by vacuum force) until pre-fixed volumes are filled in each tube.

Filled tubes (collected milk samples) are removed from the transfer device and are remained sealed and ready for direct use (feeding or testing, or can be stored in the fridge or freezer for future use/testing). For feeding, tubes are connected to the gavage feeding tubing adaptor and the rate of feeding is determined by the air flow rate into the tube.

1.-49. (canceled)

50. A device for collecting human breast milk, the device comprising at least one evacuated container having means to connect to at least one first container capable of receiving expressed human breast milk, wherein said means is optionally a flow unit permitting communication of human breast milk from the first container to the evacuated container.

51. The device according to claim 50, further comprising a filtering unit.

52. The device according to claim 50, wherein the first container, the evacuated container or both comprise at least one additive.

53. The device according to claim 52, wherein the at least one additive is selected from the group consisting of a filtration-aid agent, a homogenizing agent, a quantification aid, a preservative, a food fortifier and a therapeutic agent.

54. The device according to claim 52, wherein the at least one additive is a food grade additive.

55. The device according to claim 50, wherein the first container is adapted to hold and/or receive milk during or after breast milk expression.

56. The device according to claim 50, wherein at least part of the first container has a conical shape, a funnel-like shape, a hemisphere shape, a cylinder shape.

57. The device according to claim 50, wherein the flow unit is integrated or connected into at least part of the first container or into at least part of the evacuated container.

58. The device according to claim 50, for use in infant feeding.

59. The device according to claim 50, for use in human milk analysis.

60. A method for collecting human breast milk, the method comprising: (a) providing human breast milk in at least part of a first container of a device according to claim 50 and (b) allowing at least part of the human breast milk to flow into at least one evacuated container.

61. The method according to claim 60, wherein the human breast milk is provided into at least part of a first container during or after a milk expression process.

62. The method according to claim 61, wherein the milk expression process is manual process or using a breast pump.

63. The method according to claim 60, wherein the human breast milk is provided into at least part of a first container from a reservoir of human breast milk.

64. The method according to claim 60, wherein the flow of human breast milk from the first container into at least one evacuated container is by vacuum force.

65. The method according to claim 60, wherein the flow of human breast milk from the first container into at least one evacuated container is enabled by a flow unit.

66. The method according to claim 60, wherein the flow of human breast milk from the first container into at least one evacuated container is through a filtering unit.

67. The method according to claim 60, further comprising infant feeding.

68. The method according to claim 60, further comprising feeding an infant by connecting the evacuated container to a gavage feeding system.

69. The method according to claim 60, comprising subjecting the milk sample to human milk analysis.
